(19) United States
(12) Patent Application Publication
(10) Pub. No.: US 2019/0060860 A1
(43) Pub. Date: Feb. 28, 2019

(54) HIGH-DENSITY NUCLEIC ACID ARRAYS ON POLYESTER SUBSTRATES

(71) Applicant: Winsconsin Alumni Research Foundation, Madison, WI (US)

(72) Inventors: Matthew T. Holden, Madison, WI (US); Matthew C.D. Carter, Madison, WI (US); Lloyd M. Smith, Madison, WI (US); David M. Lynn, Middleton, WI (US)

(21) Appl. No.: 16/114,399

(22) Filed: Aug. 28, 2018

Related U.S. Application Data
(60) Provisional application No. 62/550,738, filed on Aug. 28, 2017.

(51) Int. Cl. B01J 19/00; C12Q 1/6834

(52) U.S. Cl. CPC B01J 19/0046; B01J 2219/0061; B01J 2219/00626; C12Q 1/6834

(57) ABSTRACT

Described is a method of synthesizing nucleic acids on polyester substrates and the resulting compositions of matter. The method synthesizes nucleic acids from surface hydroxyl initiation points present on the substrate surface. These surface hydroxyls are present either naturally, or as a result of a chemical treatment to cleave ester bonds on the substrate surface. The preferred polyester substrate contains PET.

### CROSS-REFERENCE TO RELATED APPLICATIONS

Priority is hereby claimed to provisional application Ser. No. 62/550,738, filed Aug. 28, 2017, which is incorporated herein by reference.

### FEDERAL FUNDING STATEMENT

This invention was made with government support under GM108727 and GM109099 awarded by the National Institutes of Health. The government has certain rights in the invention.

### BACKGROUND

Addressed arrays of surface-bound nucleic acid oligonucleotides have proven to be powerful analytical tools for the genomic sciences. Early applications of such arrays exploited their capacity to capture complementary DNA or RNA from solution to detect and quantitate the sequences present in a sample. The cost of DNA sequencing has since decreased far more rapidly than the cost of DNA synthesis which has encouraged the use of arrays as synthetic rather than analytical tools. See, for example, K. Wetterstrand (2017) “DNA Sequencing Costs: Data from the NHGRI Genome Sequencing Program (GSP)”, available online at www.genome.gov/sequencingcostsdata. While it is often now more affordable to sequence than to perform an array experiment, the parallel DNA synthesizers that fabricate arrays remain uniquely efficient means of generating complex oligonucleotide pools for gene synthesis, mutagenesis experiments, or selective enrichment for sequencing. This shift in array usage changes the considerations for the surface chemistry of the underlying array substrate. For analytical studies, it is crucial to minimize non-specific interactions between the surface and the analyte of interest. However, substrate restrictions for synthetic applications are more relaxed and typically only require that the material is robust enough to withstand the array fabrication chemistry.

Recent work on array substrates has shown that flexible sheets of poly(ethylene terephthalate) (PET) coated with multilayer polymer films can be used for the maskless array synthesis of DNA and RNA oligonucleotides. See A. H. Broderick, M. R. Lockett, M. E. Buck, Y. Yuan, L. M. Smith, D. M. Lynn (2012) Chemistry of Materials 24:938-945 and M. T. Holden, M. C. D. Carter, C.-H. Wu, J. Wolfer, E. Codner, M. R. Sussman, D. M. Lynn, L. M. Smith (2015) Analytical Chemistry 87:11420-11428. Such surfaces allow the arrays to be cut and subdivided into smaller sections more readily than arrays made on rigid substrates. The oligonucleotide attachment chemistry also proved more stable to elevated temperatures than the silyl-ether bond used in arrays synthesized on silanized glass. M. F. Phillips, M. R. Lockett, M. J. Rodesch, M. R. Shortreed, F. Cerrina, L. M. Smith (2008) Nucleic Acids Research 36, e7. However, a limitation of this past work is that the polymer multilayers used to fix hydroxyl groups on the PET are prone to delamination during the array fabrication; this process limited the length of the oligonucleotides which could be synthesized as well as the complexity of the arrays.

Maskless array synthesis (“MAS”) creates patterned nucleic acid arrays on a substrate using virtual, ultraviolet light light masks generated on a computer (in contrast to methods that use physical photolithographic masks to create the desired pattern). A reflective imaging system forms an ultraviolet image of the virtual mask on the active surface of a substrate, which is mounted in a flow cell reaction chamber connected to a DNA/RNA synthesizer. Programmed chemical coupling cycles follow light exposure, and these steps are repeated with different virtual masks to grow desired oligonucleotides in a selected pattern. See Singh-Gasson 51, Green R D, Yue Y, Nelson C, Blattner F, Sussman M R, Cerrina F. (October 1999) “Maskless fabrication of light-directed oligonucleotide microarrays using a digital micromirror array,” Nat Biotechnol. 17(10):974-8. See also U.S. Pat. No. 8,030,477, issued Oct. 4, 2011, to Cerrina et al. and U.S. Pat. No. 6,375,903, issued Apr. 23, 2002, to Cerrina et al.

Polynucleotide synthesis using phosphoramidite chemistry is well-known and extensively practiced. Because the person of ordinary skill in the biochemistry arts is familiar with this technique, it will not be described in any detail. See, for example, Reese, Colin B. (2005) “Oligo- and poly-nucleotides: 50 years of chemical synthesis,” Organic & Biomolecular Chemistry 3(21):3851.

### SUMMARY

Disclosed herein is a method to make nucleic acid arrays directly on a polyester substrate, without the need of intervening polymer layers. In the method, a polyester substrate, such as PET, is treated with a reagent that cleaves ester bonds on the polyester substrate. This yields free hydroxyl groups extending from the surface of the polyester substrate. Using conventional phosphoramidite chemistries, nucleic acids can be tethered to the substrate via the hydroxyl groups. In the preferred version of the invention, the polyester substrate is treated with a base, such as an amine-containing base, that cleaves the ester bonds in the substrate and yields hydroxyl groups. Phosphoramidite chemistry is then initiated from the hydroxyl groups to anchor nucleic acids to the substrate, thereby forming an array. The light-directed array fabrication process produces significantly less physical damage on base-treated polyester substrates than on the previously reported thin film-modified substrates. Thus, disclosed and claimed herein is a method to make nucleic acid arrays on polyester substrates, as well as the nucleic acid-modified polyester substrates so formed.

### BRIEF DESCRIPTION OF THE DRAWINGS

FIG. 1 schematically depicts the aminolysis of poly(ethylene terephthalate) to yield free hydroxyl groups which are then used as anchor points for array synthesis.

FIG. 2 illustrates the synthesis of DNA on PET substrates. Panel 6A depicts a patterned poly-(dT)₁₅ stained with Sybr Gold on PET treated with 6-amino-1-hexanol (as disclosed herein). Panel 6B depicts a fluorescence micrograph of an array on ammonia-treated PET hybridized with Texas Red- and Cy5-labelled oligonucleotides (yellow and red, respectively). Panel 6C is a graph depicting relative fluorescence intensity after hybridization of features produced on untreated PET, 6-amino-1-hexanol-treated PET, and ammonia-treated PET surfaces. Yellow bars denote signal intensity of features hybridized with Texas Red-labelled oligonucleotides and red bars denote the signal intensity of features hybridized with Cy5-labelled oligonucleotides. Error bars are +/−one standard deviation of signals from 24 different features. The different background levels evident in Panels A and B reflect the differences in non-specific binding of Sybr Gold (Panel A) and fluorescently tagged oligonucleotides (Panel B).

FIG. 3 is a graph depicting relative fluorescence signal intensity as a function of aminolysis conditions. Signal intensities represent an average of eight (8) sub-features on the array and error bars are one standard deviation. Concentrations refer to the ammonia-in-methanol solution used to treat the surfaces. Signal intensities above the 16-bit limit were calculated by adjusting values acquired at a lower scanner gain during imaging (see the Examples). The inset depicts the feature pattern used for this set of arrays.

FIG. 4 depicts the results of hybridization to arrays produced with different synthesis chemistries. “CSO” denotes 10-camphorsulfonyl)oxaziridine.

FIG. 5 is a graph depicting UV dose optimization upon the PET surfaces. An array was fabricated with features synthesized using different UV light doses to remove the 5′-NPPOC group and hybridized with complementary oligonucleotides. The normalized signal upon hybridization is charted as a function of the UV dose. The inset shows one sub-array on the surface. The UV dose increases from the top left across the row, continuing to increase again at the left-hand side of subsequent row. Normalization was conducted relative to the most intense signal within each sub-array (8 total), then averaged to produce the curve shown. Error bars represent one standard deviation. The lamp power was measured at 365 nm using a Newport 818-UV/DB photodiode sensor (Newport Corporation, Irvine, Calif., USA).

FIG. 6 is the calibration curve for the elution experiment described in the examples. The curve depicts the fluorescence signal as a function of Cy5-labelled DNA concentration in 50 μL of 8 M urea. The curve was used to measure the amount of DNA hybridized and then eluted from an array. The signals of the array eluents were measured to be 177209 and 177770 RFU which corresponded to a density of 5.84 and 5.86 pmol/cm² for silanized glass and untreated PET respectively over the 1.1 cm×1.4 cm synthesis site.

FIG. 7 is a series of identically patterned arrays fabricated on PET substrates sourced from different commercial suppliers. The arrays were fabricated according to the method disclosed herein.

FIG. 8 is a patterned array fabricated on a commercially sourced PEN substrate. The array was fabricated according to the method disclosed herein.

### DETAILED DESCRIPTION

### Definitions

As used herein, the term “array” means a population of different probe molecules that are attached to a surface such that the different probe molecules can be differentiated from each other according to relative location. Each relative location is referred to as a feature, location, or address of the array. Each address in the array can include a single copy of a probe molecule, or multiple copies of the probe molecule can be present as a population of probes at an individual address on the array. The population of probes at each address typically is homogenous, having a single species of probe, for example, multiple copies of a single nucleic acid sequence can be present at a given address (i.e., multiple nucleic acid molecules having the same sequence). This, however, is not required. In some embodiments a heterogeneous population of probes can be present at a given address. Thus, any given address in the array may include a mixture of nucleic acids having different sequences.

Neighboring addresses of the array can be discrete one from the others in that they do not physically overlap. Accordingly, the addresses can be adjacent to each other or separated by a gap. In embodiments where features are spaced apart, neighboring sites can be separated, for example, by a distance of less than 100 μm, 50 μm, 10 μm, 5 μm, 1 μm, 0.5 μm or less. The layout of the addresses on the array can also be understood in terms of center-to-center distances between neighboring addresses. This is especially useful when the individual locations are roughly circular. An array useful in the invention can have neighboring features with center-to-center spacing of less than about 100 μm, 50 μm, 10 μm, 5 μm, 1 μm, 0.5 μm or less. Furthermore, it will be understood that the distance values described above represent an average distance between neighboring addresses of an array. As such, not all neighboring addresses need to fall within the specified range unless specifically indicated to the contrary (e.g., by a specific statement that the distance constitutes a threshold distance between all neighboring addresses of an array).

The methods set forth herein can be used to make arrays of various densities. The feature density may be uniform across the entire substrate or the feature density may be non-uniform. When using maskless array synthesizers, oligonucleotide arrays with feature sizes as small as about 14 μm×14 μm and feature densities in excess of about 125,000/cm² are attainable. Using conventional photolithographic masks, nucleic acid arrays having feature densities in excess of 1 million features/cm² are possible. Larger feature sizes and smaller densities are, of course, also attainable.

As used herein, the term “surface” means a part of a support structure that is accessible to contact with reagents, beads, or analytes. The surface can be substantially flat or planar. Alternatively, the surface can be rounded or contoured. Exemplary contours that can be included on a surface are wells, depressions, pillars, ridges, channels, and the like.

Nucleic acids can be immobilized to a bead or other surface by single point covalent attachment to the surface at or near the 5′ or 3′ end of the nucleic acid. In embodiments where the nucleic acid serves as a primer, attachment is configured to leave the template-specific portion of the primer free to anneal to its cognate template and the 3′ hydroxyl group free for primer extension. Any suitable covalent attachment means known in the art may be used for this purpose. The chosen attachment chemistry will depend on the nature of the solid support, and any derivatization or functionalization applied to it. The primer itself may include a moiety, which may be a non-nucleotide chemical modification, to facilitate attachment. In a particular embodiment, the primer may include a nucleophile located at the 5′ end.

Generally, conventional phosphoramidite chemistry is used for affixing a first nucleoside to the surface and then assembling the remainder of the desired DNA oligonucleotide. When making DNA oligonucleotides, the process proceeds in a well-known, four-step synthesis cycle. In the first step, detritylation, the 5′-DMT protecting group is removed from the first, solid-support-linked nucleoside. In the second step, coupling, the free 5′-OH of the first, solid-support-linked nucleoside attacks the phosphorus of the incoming second nucleoside, displacing an activating group. In the third step, capping, solid-support-linked nucleosides having an unreacted 5′-OH are acetylated. This prevents elongation of sequences with deletion mutations. In the fourth step, oxidation, the unstable phosphite triester is converted to a stable phosphate triester (which allows the next cycle to proceed to step 1, detritylation of the second nucleotide). The cycle then begins again with the next nucleotide.

The term “polyester” as used herein refers to a class of polymers that contain repeating ester functional groups in the backbone of the polymer. The term “polyester” explicitly includes, without limitation, polyesters such as poly(ethylene terephthalate) (“PET”):

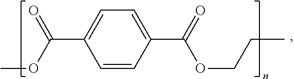

poly(trimethylene terephthalate) (“PTT”):

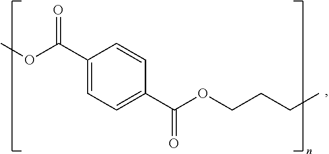

poly(butylene terephthalate) (“PBT”):

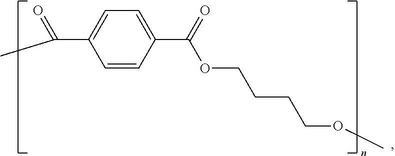

poly(ethylene naphthalate) (also known as poly(ethylene 2,6-naphthalate) (“PEN”):

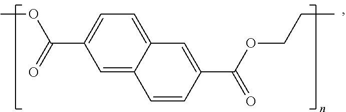

and the like. A host of suitable polyester substrates are available from a large number of national and international suppliers, including Toray Plastics America (Kingstown, R.I., USA), DuPont Teijin Films (Chester, Va., USA), BASF (Ludwisgshafen, Germany), and many others.

The molecular mass of the polyester chosen may be determined using any number of well-known means, including gel permeation chromatography (GPC) and intrinsic viscosity measurements using commercial devices. Weight-average and number-average molecular weight calculations, as well as comparison to external molecular weight markers, are well known. See, for example, ASTM-D4001: “Standard Test Method for Determination of Weight-Average Molecular Weight of Polymers by Light Scattering” and Farah et al. “8—Molecular Weight Determination of Polyethylene Terephthalate,” in Poly(Ethylene Terephthalate) Based Blends, Composites and Nanocomposites, pp. 143-165. P. M. Visakh Tomsk and Mong Liang, Eds., copyright © 2015 Elsevier Inc., 978-0-323-31306-3 GPC can be used to measure the polydispersity index of any given polyester, as well as its viscosity molecular weight (My). See ASTM D2857, “Standard Practice for Dilute Solution Viscosity of Polymers.” Rather than molecular mass per se, intrinsic viscosity is widely used in the polyester industry as a proxy measurement to describe the extent of polymerization (and hence the molecular mass) of any given polyester resin. Briefly, intrinsic viscosity measures a solute's contribution to the viscosity of a solution—in this case the dissolved polyester's contribution to the viscosity of a known concentration of the polyester in a suitable solvent. Intrinsic viscosity is defined as

$$[ η ] = {lim}_{φ \to 0} \frac{η - {η}_{0}}{{η}_{0} φ}$$

wherein η₀ is the viscosity in the absence of the solute and ϕ is the volume fraction of the solute in the solution. The volume fraction ϕ is conventionally given in g/dL. As defined, the intrinsic viscosity [η] is a dimensionless number because it is a limit that extrapolates the volume fraction of the polymer solute to zero. Intrinsic viscosity is dependent upon the length of the polymer chains. The longer the polymer chains, the more entanglements between chains and therefore the higher the intrinsic viscosity.

The intrinsic viscosity range of essentially all commercial grades of PET, PTT, PBT, and PEN range from about 0.40 to about 2.00 (see Gupta, V. B. and Bashir, Z. (2002) Chapter 7, p. 320 in Fakirov, Stoyko (ed.) Handbook of Thermoplastic Polyesters, Wiley-VCH, Weinheim, ISBN 3-527-30113-5). All commercially available polyesters can be used in the present method. Preferred, however, are polyesters having an intrinsic viscosity of about 0.50 to about 1.0. Film-grade and bottle-grade PET, having an intrinsic viscosity of about 0.6 to about 1.0, is very widely available commercially.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 1 to 9, from 3.6 to 4.6, from 3.5 to 9.9, and so forth.

All references to singular characteristics or limitations of the present invention shall include the corresponding plural characteristic or limitation, and vice-versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The methods of the present invention can comprise, consist of, or consist essentially of the essential elements and limitations of the method described herein, as well as any additional or optional ingredients, components, or limitations described herein or otherwise useful in synthetic organic chemistry.

### The Method

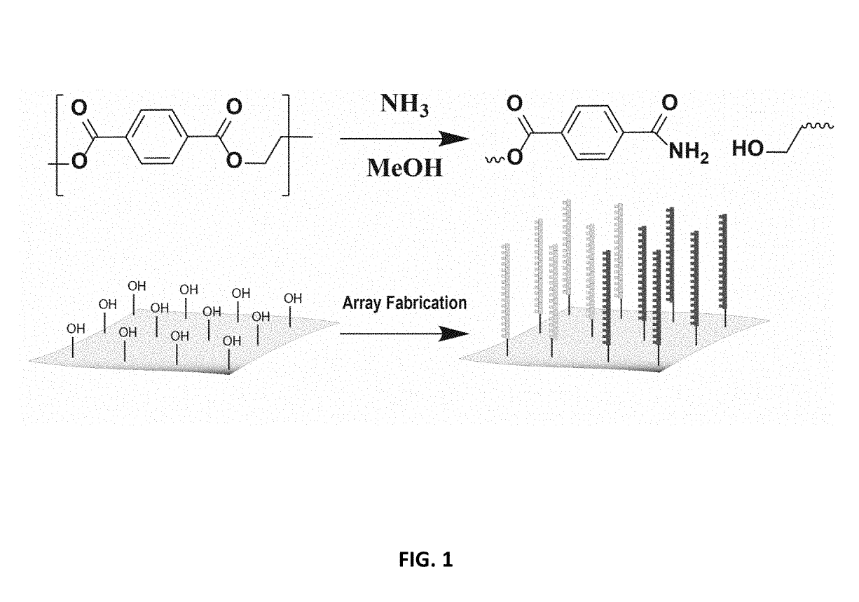

The first step of the method, while preferred, is optional. This first step is to create an increased density of free hydroxyl groups on the surface of the polyester substrate. Depending upon the polymerization route chosen, many untreated polyesters have a low density of free hydroxyl groups already. It is much preferred, however, to use a polyester substrate having a far greater density of free hydroxyl groups. In the discussion that follows, reference will be made to a PET substrate. This is for clarity and brevity only. Any of the polyesters noted above will perform similarly. PET is readily functionalized using aminolysis. The amine cleaves the polymer at the ester linkage to generate a free hydroxyl. See FIG. 1, which depicts the aminolysis reaction in the top panel. The bottom panel depicts schematically how the resulting free hydroxyl groups are used to fabricate an array on the treated PET substrate.

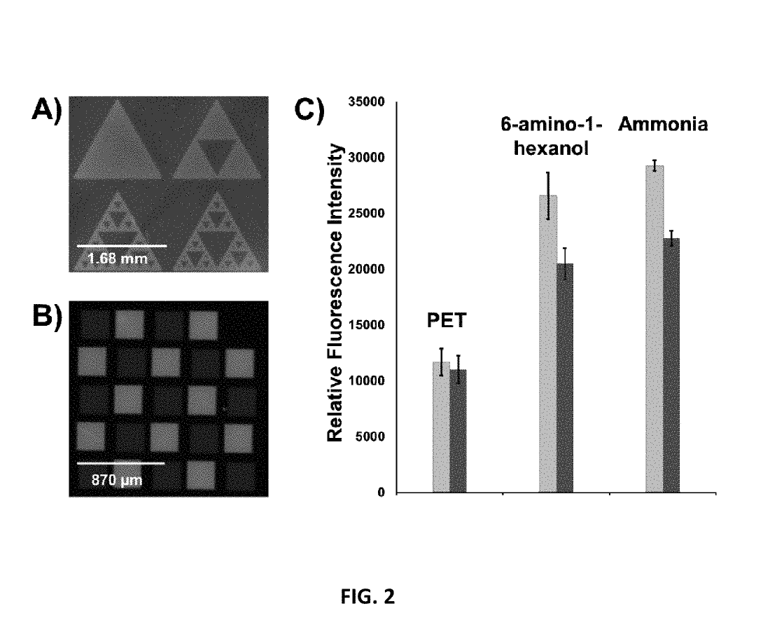

To test this reaction for producing array substrates, a thin sheet of PET was treated with 6-amino-1-hexanol in methanol. A custom-built maskless array synthesizer as described in S. Singh-Gasson, R. D. Green, Y. Yue, C. Nelson, F. Blattner, M. R. Sussman, F. Cerrina (1999) Nat Biotech 17:974-978 was then used to synthesize a poly-(dT)15 oligonucleotide. FIG. 2, panel A shows the results of staining this surface with Sybr Gold, indicating that DNA was synthesized only at the desired locations on the polymer.

The effect of the aminolysis conditions was then examined in more detail. First, the 6-amino-1-hexanol functionalization was compared to treatment with ammonia in methanol. PET surfaces were treated with either ammonia or 6-amino-1-hexanol of equal molarity and the resultant arrays were hybridized with fluorescently-labelled oligonucleotides. An example array is shown in FIG. 2, panel B. The intensity of the fluorescence signals in panels A and B of FIG. 2 are depicted in FIG. 2, panel C. The intensities are relative to an untreated PET surface. The similar signal intensities of the treated substrates establish that it is not crucial to use a hydroxyl-bearing amine to functionalize the surface. Whether the synthesis occurs preferentially from the hydroxyl of the 6-amino-1-hexanol or the PET backbone was not studied further. It is likely that the benefit of using a hydroxyl-containing amine is offset by a lower reactivity relative to ammonia (data not shown). It may also be difficult in practice for a hybridization assay to detect both the expected hydroxyls if they remain in close enough proximity to hinder the DNA synthesis or duplex formation. Notably, it was also possible to synthesize DNA on untreated PET, albeit at a lower density. This results from residual hydroxyl end groups remaining from the polymerization process or those that accrue from various modes of degradation.

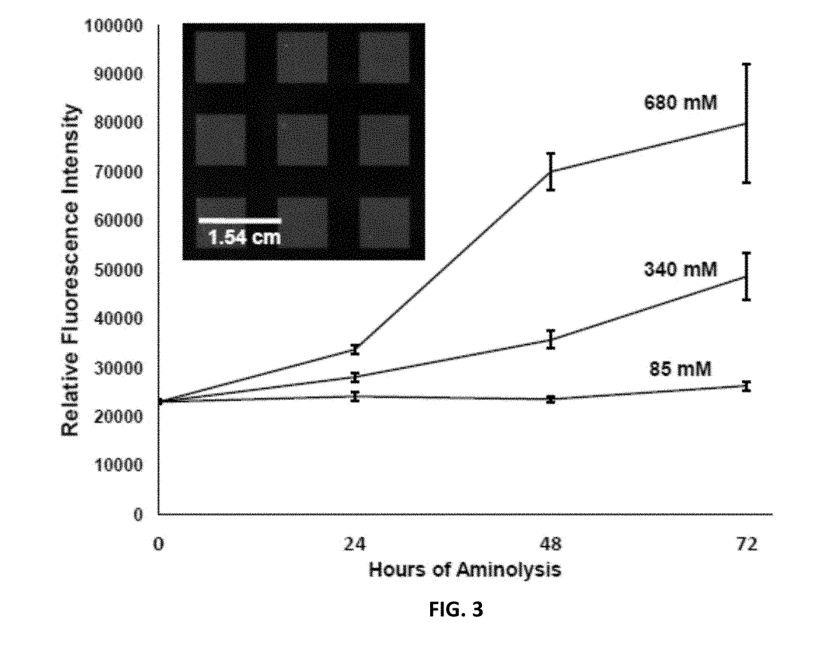

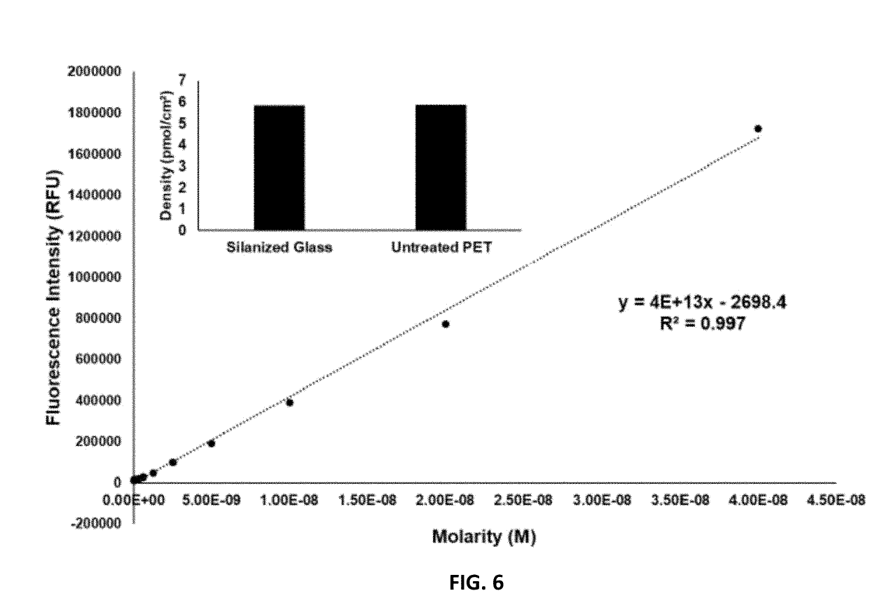

A functional estimate of the new synthesis sites exposed by the aminolysis was then made using hybridization experiments. Arrays containing a single sequence were fabricated upon PET treated with various ammonia concentrations and hybridized with fluorophore-labelled oligonucleotides. FIG. 3 depicts the relationship between the resultant signal intensity and the treatment conditions. This ability to control surface oligonucleotide density allows the surfaces to be optimized for either the synthesis of long DNA sequences or for yield, similar to the optimization of the controlled pore glass supports employed for standard solid-phase DNA synthesis. Wash-off experiments suggest that the oligonucleotide density of untreated PET is similar to that of silanized glass (˜6 pmol/cm², a typical site density for array fabrication, see FIG. 6) See M. R. Lockett, M. F. Phillips, J. L. Jarecki, D. Peelen, L. M. Smith, Langmuir 2008, 24, 69-75.

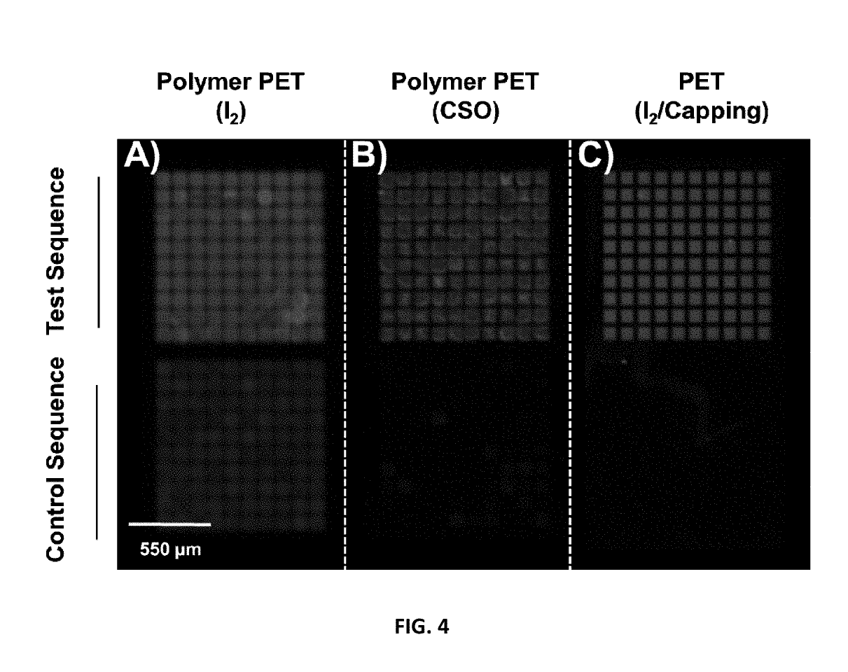

Covalently attaching the array oligonucleotides to the substrate material reduces the points at which chemical degradation can occur. The functional stability of the PET substrate was assessed by synthesizing arrays with oligonucleotides of greater length than had been made previously upon flexible substrates. FIG. 4 shows the results of hybridization of a fluorescently-labelled oligonucleotide complementary to the 18 nt termini of 88 nt features. (The sequence information is given in Table 3.) The upper half of each of panels A, B, and C in FIG. 4 depicts a 100-feature sub-array where fluorescence is expected; the lower half of each panel depicts the region where a control sequence was synthesized and no fluorescence should be observed. Each array was fabricated under a different set of conditions. Those depicted in FIG. 4, panels A and B were synthesized upon PET coated with multiple polymer bilayers as previously described and differ only in the oxidizer used during the phosphoramidite cycle: In panel A, iodine, pyridine, and water in tetrahydrofuran (THF) was used as the oxidizer; in Panel B, 0.5 M (10-camphorsulfonyl)oxaziridine (CSO) in acetonitrile was used as the oxidizer.

Panels A and B of FIG. 4 exhibit surface defects and high non-specific binding that complicate potential analytical applications. The oligonucleotides on the surface may be usable for synthetic applications, but the poor feature definition suggests a loss of precision during the photodeprotection step of the synthesis. This would reduce the sequence fidelity and the ability to miniaturize the arrays. (C. Agbavwe, C. Kim, D. Hong, K. Heinrich, T. Wang, M. M. Somoza, J. Nanobiotechnol. 2011, 9, 57-57 and P. B. Garland, P. J. Serafinowski, Nucleic Acids Res. 2002, 30, e99-e99.) In contrast, the array in FIG. 4, panel C, which was made according to the present method, shows no evidence of substrate degradation and the spatial separation between features is preserved. The array shown in FIG. 4, panel C was synthesized upon ammonia-treated PET using iodine/pyridine/water in THF as an oxidizer and included a step of acetylation to cap failed sequences during synthesis. As noted previously, acetylation is commonly used in solid-phase phosphoramidite chemistry to truncate failed couplings so that sequences which would contain deletions can be removed from full-length products. (See, for example, S. Ma, I. Saaem, J. Tian (2012) Trends in biotechnology 30:147-154.) In the light-directed format used here, acetylation also acts to reduce insertion errors. This achievement solves an unmet need in the field because it had not been possible to use an acetylation step on previous modified PET-based substrates as the solvents involved were too damaging to the substrate surface.

This work establishes that polyesters such as poly(ethylene terephthalate) can be used as a substrate for DNA synthesis. The primary anchor for the first phosphoramidite coupling is the terminal hydroxyl of the polymer chains comprising the substrate. While amine treatments were explored here, any base treatment strong enough to cleave the PET ester will achieve a similar result. Synthesizing oligonucleotides directly from the substrate material improves resistance to the chemical processing needed for high-density array synthesis while preserving the desirable aspects of a flexible substrate. These findings show that base-treated PET is a simple yet attractive substrate choice for parallel DNA synthesis. The base treatments are applicable to other polyesters, many of which are used as 3D printing materials. 3D printed array substrates could include wells for gene assembly or other enzymatic reactions, integrate with existing fluidics platforms, be rendered electrically conductive, or be divided into small sub-arrays which could be manipulated with magnets.

### EXAMPLES

The following Examples are included solely to provide a more complete disclosure of the methods and materials disclosed and claimed herein. The Examples are not intended to limit the scope of the claims.

### Materials

6-Amino-1-hexanol, 2 M ammonia in methanol (MeOH), methanol, 5′-nitrophenylpropyloxycarbonyl (NPPOC)-protected phosphoramidites, ethylenediamine, ethanol (for array deprotection), dimethyl sulfoxide (DMSO), 2,6-lutidine, imidazole, pyridine, 1-methylimidazole, 20×SSPE buffer (20 mM ethylenediaminetetraacetic acid and 2.98 M NaCl in 200 mM phosphate buffer, pH 7.4), 384 well plates, branched poly(ethylenimine) (MW ˜25,000), acetone, ethyl acetate, 2,2′-azobis(2-methylpropionitrile), tetrahydrofuran (THF), polyethylene glycol sorbitan monolaurate (“TWEEN 20”-brand), and fluorophore-labelled oligonucleotides were purchased from Sigma-Aldrich (St. Louis, Miss., USA). 5′-Benzoyl-2-(2-nitrophenyl)propoxycarbonyl (Bz-NPPOC) protected phosphoramidites were purchased from Orgentis Chemicals GmbH (Gaterslaben, Germany). SuperClean glass substrates were purchased from Arrayit (Sunnyvale, Calif.). N-(3-triethoxysilylpropyl)-4-hydroxybutyramide was purchased from Gelest (Morrisville, Pa.). 2-Dimethylaminopyridine and D-glucamine were purchased from Tokyo Chemical Industry Co. LTD (Tokyo, Japan). 3% Dichloroacetic acid in toluene, anhydrous acetonitrile (ACN), 5% phenoxyacetic anhydride in THF, 0.5 M (1S)-(+)-(10-camphorsulfonyl)oxaziridine (CSO) in acetonitrile, and 0.25 M dicyanoimidazole in acetonitrile were purchased from Glen Research (Sterling, Va., USA). Dimethoxytrityl (DMT)-protected polyethylene glycol (MW 2000) phosphoramidite and oxidizer (0.02 M iodine in THF/pyridine/H₂O) were purchased from ChemGenes Corporation (Wilmington, Mass., USA). Gene Frame gaskets were purchased from Thermo Fisher Scientific (Madison, Wis., USA). Dry packs were purchased from BioAutomation (Irving, Tex., USA). PET film (0.1 mm thick) was purchased from McMaster Carr (Elmhurst, Ill., USA), TEKRA, a division of EIS, Inc. (New Berlin, Wis., USA), and ePlastics (San Diego, Calif., USA). PEN film was purchased from TEKRA Methanol (ACS grade, for bilayer fabrication) was purchased from Avantor Performance Materials (Center Valley, Pa., USA). Ethanol (200 proof, for bilayer fabrication) was purchased from Decon Laboratories (King of Prussia, Pa., USA). 2-Vinyl-4,4-dimethylazlactone was a gift from Dr. Steven M. Heilmann (3M Corporation, Minneapolis, Minn., USA). Fluorophore-labelled oligonucleotides were also purchased from Integrated DNA Technologies (Coralville, Iowa, USA). Poly(2-vinyl-4,4-dimethylazlactone) (PVDMA) was synthesized as described in M. E. Buck, S. C. Schwartz, D. M. Lynn, (2010) “Chemistry of materials: a publication of the American Chemical Society,” 22:6319-6327.

### Methods

### Surface Treatment

PET was cut into slide-sized sections and placed into 50 mL conical tubes filled with 40 mL of anhydrous methanol containing either dissolved 6-amino-hexanol or ammonia. The solutions were incubated at 37° C. overnight unless otherwise noted. After incubation, the PET was rinsed in methanol and dried under a nitrogen stream prior to array fabrication. Polymer-bilayer-coated PET and silanized glass slides was prepared as described in M. T. Holden, M. C. D. Carter, C.-H. Wu, J. Wolfer, E. Codner, M. R. Sussman, D. M. Lynn, L. M. Smith (2015) Analytical Chemistry 87:11420-11428 and M. F. Phillips, M. R. Lockett, M. J. Rodesch, M. R. Shortreed, F. Cerrina, L. M. Smith, Nucleic Acids Research 2008, 36, e7-e7 See Table 1.

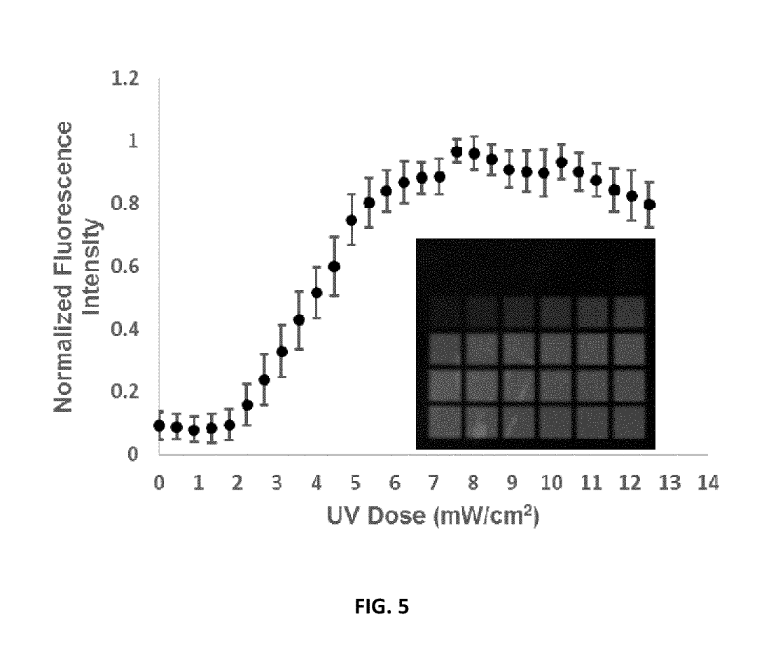

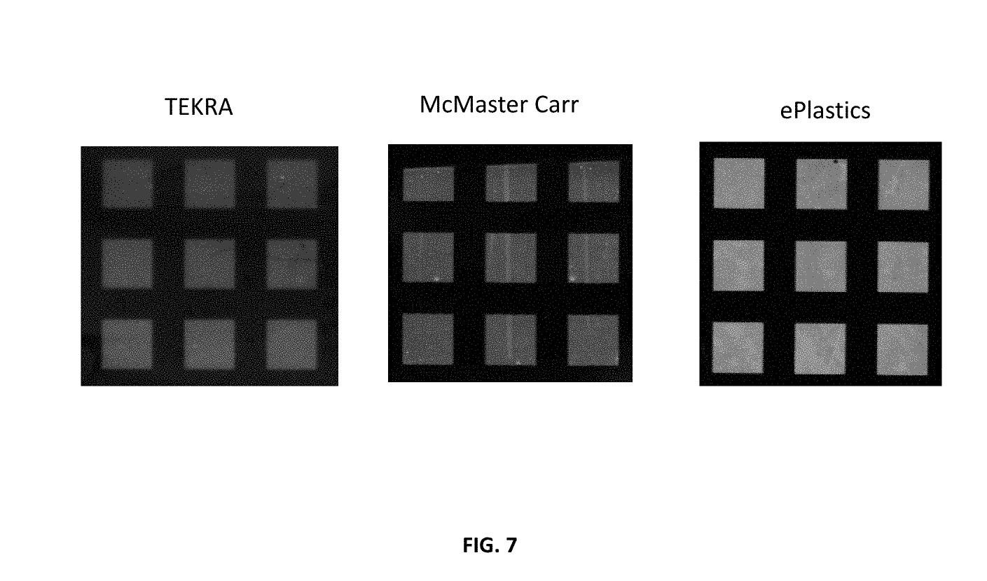

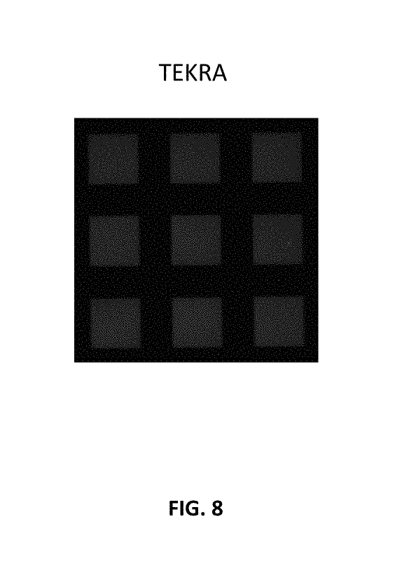

TABLE 1
Aminolysis Conditions.

| Figure No. | Amine | Conditions |
|---|---|---|
| FIG. 2, panel A | 6-amino-1-hexanol | 680 mM, 24 hrs. at 37° C. |
| FIG. 2, panel B | Ammonia in MeOH | 680 mM, 24 hrs at 37° C. |
| FIG. 3, graph | Ammonia in MeOH | As described in text, at 37° C. |
| FIG. 3, inset | Ammonia in MeOH | 85 mM, 24 hrs at 37° C. |
| FIG. 4, panel C | 6-amino-1-hexanol | 680 mM, 24 hrs. at 37° C. |
| FIG. 5 | 6-amino-1-hexanol | 170 mM, 24 hrs. at 37° C. |
| FIGS. 7 and 8 | Ammonia in MeOH | 340 mM, 24 hrs, at 37° C. |

### Array Fabrication

The arrays were fabricated on a custom-built maskless array synthesizer using the same process and chemistry as described in the literature. See M. T. Holden, M. C. D. Carter, C.-H. Wu, J. Wolfer, E. Codner, M. R. Sussman, D. M. Lynn, L. M. Smith (2015) Analytical Chemistry 87:11420-11428; and C.-H. Wu, M. T. Holden, L. M. Smith (2014) Angewandte Chemie (International ed. in English) 53:13514-13517. The UV dose for 5′-NPPOC removal (7.5 J/cm²) was determined by measuring hybridization intensity to array features synthesized using different exposure times. Arrays were deprotected in a 50:50 v/v mix of ethylenediamine:ethanol for 60 minutes. The UV dose data is shown in FIG. 5. A summary of the chemical synthesis conditions and oligonucleotides produced are shown in Tables 2 and 3, respectively.

TABLE 2
Array Synthesis Conditions.

| | | | | Oxidation | Capping |
|---|---|---|---|---|---|
| FIG. | Activator | Oxidizer | Oxidation Time | Pattern | (Yes/No) |
| FIG. 2, | DCI | Iodine/THF/Pyridine/Water | 60 Expedite | Every 5 NPPOC- | N |
| panel A | | | pulses/NPPOC | phosphoramidite | |
| | | | oxidation cycle | couplings | |
| FIG. 2, | DCI | Iodine/THF/Pyridine/Water | 60 Expedite | Every 3 NPPOC | N |
| panel B | | | pulses/NPPOC | couplings | |
| | | | oxidation cycle | | |
| FIG. 3 | DCI | Iodine/THF/Pyridine/Water | 60 Expedite | Every 5 NPPOC- | N |
| | | | pulses/NPPOC | phosphoramidite | |
| | | | oxidation cycle | couplings | |
| FIG. 4, | DCI | Iodine/THF/Pyridine/Water | 60 Expedite | Every 3 NPPOC- | N |
| panel A | | | pulses/NPPOC | phosphoramidite | |
| | | | oxidation cycle | couplings and | |
| | | | | every DMT- | |
| | | | | phosphoramidite | |
| | | | | coupling | |
| FIG. 4, | DCI | CSO in ACN | 4 minutes | Every 3 NPPOC- | N |
| panel B | | | | phosphoramidite | |
| | | | | couplings and | |
| | | | | every DMT- | |
| | | | | phosphoramidite | |
| | | | | coupling | |
| FIG. 4, | DCI | Iodine/THF/Pyridine/Water | 60 Expedite | Every 3 NPPOC- | Y |
| panel C | | | pulses/NPPOC | phosphoramidite | |
| | | | oxidation cycle | couplings and | |
| | | | | every DMT- | |
| | | | | phosphoramidite | |
| | | | | coupling | |
| FIG. 5 | DCI | Iodine/THF/Pyridine/Water | 60 Expedite | Every 3 NPPOC | N |
| | | | pulses/NPPOC | couplings | |
| | | | oxidation cycle | | |
| FIG. 6 | DCI | Iodine/THF/Pyridine/Water | 60 Expedite | Every 5 Bz- | N |
| | | | pulses/Bz- | NPPOC- | |
| | | | NPPOC | phosphoramidite | |
| | | | oxidation cycle | couplings | |
| FIGS. 7 | DCI | Iodine/THF/Pyridine/Water | 60 Expedite | Every 5 Bz- | N |
| and 8 | | | pulses/NPPOC | NPPOC- | |
| | | | oxidation cycle | phosphoramidite | |
| | | | | couplings; | |
| | | | | NPPOC used for | |
| | | | | McMaster Carr | |
| | | | | sample | |

TABLE 3
Sequences of Oligonucleotides Synthesized on the Arrays.

| Figure | Feature | DNA Sequence (3′-5′) |
|---|---|---|
| FIG. 2, | 1 | TTTTTTTTTTTTTTT (SEQ. ID. NO: 1) |
| panel A | | |
| FIGS. 2, | 1 | TTTTTTTTTTTAGTCTTGAGTGGACAATC (SEQ. ID. NO: 2) |
| panel B | 2 | TTTTTTTTTTTCGGCTACTGGACGTTCTCA (SEQ. ID. NO: 3) |
| FIGS. 3, | 1 | TTTTTTTTTTTAGTCTTGAGTGGACAATC (SEQ. ID. NO: 4) |
| 7, and 8 | | |
| FIG. 4, | Test | T/PEG2k/TTTTTTTTTTTTTTTTCCTGTGCCGCTTTCGGCTACTGG |
| panels | | ACGTTCTCATTATTGAAACGTTGTCACCTAGTCTTGAGTGGAC |
| A-C | | AATC (SEQ. ID. NO: 5) |
| | Control | T/PEG2k/TTTTTTTTTTTTTTTTCCTGTGCCGCTTTTGAGAACGT |
| | | CCAGTAGCCGTGGTGACAACGTTTCAATATGATTGTCCACTCA |
| | | AGACT (SEQ. ID. NO: 6) |
| FIG. 6 | All | TTTTTTTTTTTCGGCTACTGGACGTTCTCA (SEQ. ID. NO: 7) |

“PEG2k” denotes a dimethoxytrityl-protected polyethylene glycol phosphoramidite (MW ~ 2000).

### Array Hybridization

Arrays were hybridized with 1 μM solutions of fluorescently-labelled oligonucleotides in 4×SSPE buffer containing 0.1% TWEEN 20. After the solution was placed on the surface, the arrays were incubated in a humid chamber for 30 minutes or more at 37° C. They were then rinsed at room temperature in 0.5×SSPE buffer prior to imaging.

TABLE 4
Oligonucleotides used for Hybridization
Experiments.

| Label | Sequence (5′-3′) | Usage |
|---|---|---|
| 3′-Texas Red | GCCGATGACCTGCAAGAGT | FIG. 2, panel B |
| | (SEQ. ID. NO: 8) | and FIG. 5 |
| 5′-Cy5 | TCAGAACTCACCTGTTAG | FIG. 2, panel B, |
| | (SEQ. ID. NO: 9) | FIG. 3, |
| | | FIG. 4, panels |
| | | A-C, and FIGS. |
| | | 6, 7, and 8 |

### Elution Experiment

Arrays containing a single sequence were hybridized with a fluorophore-labelled oligonucleotide. They were then rinsed in 0.5×SSPE and dehybridized in 2 mL of 8 M urea. The fluorescence intensity of the urea solution was measured against a calibration curve of the same fluorophore-labelled oligonucleotide (FIG. 6). Readings were acquired with a Perkin Elmer Envision 2100 Multilabel Reader (Waltham, Mass., USA) using a filter set for Cy5.

### Image Acquisition, Analysis, and Figure Preparation

The arrays were placed onto a glass microscope slide and, in cases where a Gene Frame gasket was used, a cover slip was placed over the gasket to keep the features wetted. However, it was found that the background signal on the arrays was generally lower when the arrays were placed under a cover slip without a gasket. In cases where hybridization intensities between different arrays were being compared (FIG. 2, panel C and FIG. 3), gasket use was kept consistent throughout the entire set. Images were acquired on a GeneTac UC 4×4 scanner (Genomic Solutions Inc., Ann Arbor, Mich., USA). Processing was conducted with ImageJ software (NIH, Bethesda, Md., USA; https://imagej.nih.gov/ij/) on the 16-bit grayscale files produced by the scanner. Table 5 presents the acquisition parameters and the false-color palettes used in the figures. In FIG. 3, values above the 16-bit limit were calculated by rescanning the arrays at a lower gain and adjusting by the percent change observed at a common reference point on the high and low gain images.

TABLE 5
Image Acquisition Parameters.

| | | | | False- |
|---|---|---|---|---|
| FIG. | Channel | Resolution | Scanner Gain | Coloring |
| FIG. 2, | Cy3 | 5 μm/pixel | 40 | Green |
| panel A | | | | |
| FIG. 2, | Texas Red | 10 μm/pixel | 40 | Yellow |
| panel B | | | | |
| | Cy5 | 10 μm/pixel | 30 | Red |
| FIG., 3 (inset) | Cy5 | 20 μm/pixel | 28 | Green |
| FIG., 4, | Cy5 | 10 μm/pixel | 32 | Red |
| panel A | | | | |
| FIG., 4, | Cy5 | 10 μm/pixel | 32 | Red |
| panel B | | | | |
| FIG., 4, | Cy5 | 10 μm/pixel | 32 | Red |
| panel C | | | | |
| FIG. 5, | Texas Red | 20 μm/pixel | 42 | Green |
| inset | | | | |
| FIG. 7, left- | Cy5 | 20 μm/pixel | 38 | Green |
| hand panel | | | | |
| FIG. 7, | Cy5 | 20 μm/pixel | 28 | Green |
| middle panel | | | | |
| FIG. 7, right- | Cy5 | 20 μm/pixel | 24 | Green |
| hand panel | | | | |
| FIG. 8 | Cy5 | 20 μm/pixel | 38 | Green |

### Commercial Polyester Substrates

The array fabrication strategy disclosed herein was validated on commercial PET samples from various vendors. Identical arrays were fabricated on commercial PET substrates obtained from TEKRA, McMaster Can and ePlastics. The arrays were fabricated as described in the Examples. The results are shown in FIG. 7. The right-hand array was fabricated on a PET substrate obtained from TEKRA. The middle array was fabricated on a PET substrate obtained from McMaster Carr. The right-hand array was fabricated on a PET substrate obtained from ePlastics. While all substrates resulted in successful arrays, there was a wide variation in signal intensity upon hybridization. but may arise from differences in the distribution of end groups, molecular weights, or microscale morphology of the surface. The generality of the approach was also tested on a poly(ethylene) napthalate (PEN) sample, to demonstrate the method does work using other polyester substrates. The results here are shown in FIG. 8. Shown in the figure is an array fabricated according to the present method, using a PEN substrate obtained commercially from TEKRA.

What is claimed is:

1. A method of affixing a nucleic acid to a polyester substrate, the method comprising:
(a) covalently bonding nucleic acids to at least a portion of free hydroxyl groups on a surface of a polyester substrate.

2. The method of claim 1, wherein step (a) comprises creating an addressed array of nucleic acids on the substrate.

3. The method of claim 1, wherein the polyester substrate comprises a polyester selected from poly(ethylene terephthalate) (“PET”), poly(trimethylene terephthalate) (“PTT”), poly(butylene terephthalate) (“PBT”), and poly(ethylene naphthalate) (“PEN”).

4. The method of claim 1, wherein the polyester substrate comprises PET.

5. The method of claim 1, further comprising, before step (a), cleaving ester bonds in the polyester substrate to create additional free hydroxyl groups on a surface of the polyester substrate.

6. The method of claim 5, wherein the ester bonds in the polyester substrate are cleaved by treating the polyester substrate with a base.

7. The method of claim 5, wherein the ester bonds in the polyester substrate are cleaved by treating the polyester substrate with an amine-containing reagent.

8. The method of claim 5, wherein the ester bonds in the polyester substrate are cleaved by treating the polyester substrate with ammonia or 6-amino-1-hexanol.

9. The method of claim 5, wherein step (a) comprises synthesizing poly(nucleic acids) step-wise that are covalently bonded to the surface using as initiation points at least a portion of the free hydroxyl groups.

10. The method of claim 9, wherein the poly(nucleic acids) are synthesized via phosphoramidite chemistry.

11. The method of claim 10, wherein step (a) comprises creating an addressed array of poly(nucleic acids) on the substrate.

12. The method of claim 10, wherein the polyester substrate comprises a polyester selected from poly(ethylene terephthalate) (“PET”), poly(trimethylene terephthalate) (“PTT”), poly(butylene terephthalate) (“PBT”), and poly(ethylene naphthalate) (“PEN”).

13. The method of claim 10, wherein the polyester substrate comprises PET.

14. The method of claim 10, further comprising, before step (a), cleaving ester bonds in the polyester substrate to create additional free hydroxyl groups on a surface of the polyester substrate.

15. The method of claim 14, wherein the ester bonds in the polyester substrate are cleaved by treating the polyester substrate with a base.

16. The method of claim 14, wherein the ester bonds in the polyester substrate are cleaved by treating the polyester substrate with an amine-containing reagent.

17. The method of claim 14, wherein the ester bonds in the polyester substrate are cleaved by treating the polyester substrate with ammonia or 6-amino-1-hexanol.

18. A method of affixing a nucleic acid to a polyester substrate, the method comprising:
(a) cleaving ester bonds in a polyester substrate to create free hydroxyl groups on a surface of the polyester substrate; and
(b) synthesizing poly(nucleic acids) that are covalently bonded to the surface using as initiation points at least a portion of the free hydroxyl groups created in step (a).

19. The method of claim 18, wherein in step (a) the ester bonds in the polyester substrate are cleaved by treating the polyester substrate with a base.

20. The method of claim 18, wherein in step (a) the ester bonds in the polyester substrate are cleaved by treating the polyester substrate with an amine-containing reagent.

21. The method of claim 18, wherein in step (a) the ester bonds in the polyester substrate are cleaved by treating the polyester substrate with ammonia or 6-amino-1-hexanol.

22. The method of claim 18, wherein step (a) comprises synthesizing poly(nucleic acids) step-wise that are covalently bonded to the surface using as initiation points at least a portion of the free hydroxyl groups.

23. The method of claim 22, wherein the poly(nucleic acids) are synthesized via phosphoramidite chemistry.

24. The method of claim 18, wherein step (b) comprises creating an addressed array of nucleic acids on the substrate.

25. The method of claim 18, wherein the polyester substrate comprises a polyester selected from poly(ethylene terephthalate) (“PET”), poly(trimethylene terephthalate) (“PTT”), poly(butylene terephthalate) (“PBT”), and poly(ethylene naphthalate) (“PEN”).

26. The method of claim 18, wherein the polyester substrate comprises PET.

27. A composition of matter comprising a polyester substrate having covalently bonded thereto, in the absence of any intervening polymeric layer, at least one nucleic acid molecule.

28. The composition of matter of claim 27, wherein the polyester substrate comprises a polyester selected from poly(ethylene terephthalate) (“PET”), poly(trimethylene terephthalate) (“PTT”), poly(butylene terephthalate) (“PBT”), and poly(ethylene naphthalate). (“PEN”).

29. The composition of matter of claim 27, wherein the polyester substrate comprises PET.

30. The composition of matter of claim 27, comprising an addressed array of nucleic acids on the substrate.


SEQUENCE LISTING (7 sequences)

SEQ ID NO: 1 - DNA, 15 bp, Artificial Sequence
  Artificial DNA sequence fabricated step-wise from individual nucleotide bases.
ttttttttttttttt

SEQ ID NO: 2 - DNA, 29 bp, Artificial Sequence
  Artificial DNA sequence fabricated step-wise from individual nucleotide bases.
tttttttttttagtcttgagtggacaatc

SEQ ID NO: 3 - DNA, 30 bp, Artificial Sequence
  Artificial DNA sequence fabricated step-wise from individual nucleotide bases.
tttttttttttcggctactggacgttctca

SEQ ID NO: 4 - DNA, 29 bp, Artificial Sequence
  Artificial DNA sequence fabricated step-wise from individual nucleotide bases.
tttttttttttagtcttgagtggacaatc

SEQ ID NO: 5 - DNA, 87 bp, Artificial Sequence
  Artificial DNA sequence fabricated step-wise from individual nucleotide bases.
  misc_feature (1)..(1): Residue 1 is modified to include a dimethoxytrityl-protected polyethylene glycol phosphoramidite
tttttttttttttttttcctgtgccgctttcggctactggacgttctcattattgaaacgttgtcacctagtcttgagtggacaatc

SEQ ID NO: 6 - DNA, 87 bp, Artificial Sequence
  Artificial DNA sequence fabricated step-wise from individual nucleotide bases.
  misc_feature (1)..(1): Position 1 is modified to include a dimethoxytrityl-protected polyethylene glycol phosphoramidite.
tttttttttttttttttcctgtgccgcttttgagaacgtccagtagccgtggtgacaacgtttcaatatgattgtccactcaagact

SEQ ID NO: 7 - DNA, 30 bp, Artificial Sequence; identical to SEQ ID NO: 3
  Artificial DNA sequence fabricated step-wise from individual nucleotide bases.